(19) United States
(12) Patent Application Publication
(10) Pub. No.: US 2010/0160939 A1
(43) Pub. Date: Jun. 24, 2010

(54) SYSTEMS, APPARATUSES, AND METHODS FOR CARDIOVASCULAR CUTTING DEVICES AND VALVES

(75) Inventor: Peter N. Braido, Maple Grove, MN (US)

Correspondence Address: HOLLAND & HART LLP, 60 E. South Temple, Suite 2000, P.O. Box 11583, Salt Lake City, UT, 84110, US

(73) Assignee: St. Jude Medical, Inc., St. Paul, MN (US)

(21) Appl. No.: 12/340,431

(22) Filed: Dec. 19, 2008

(51) Int. Cl. A61B 17/32

(52) U.S. Cl. 606/170

(57) ABSTRACT

A system comprising a tube with a first end dimension to be positioned against a cardiovascular organ. The system may comprise a cutting member adapted to cut an opening in the cardiovascular organ. The system may also comprise a first valve positioned inside the tube. The first valve may be adapted to open to allow the cutting member to pass through the first valve to the cardiovascular organ. The first valve may be adapted to close after the cardiovascular coring device is retracted through the first valve. A cardiovascular coring apparatus is also disclosed. The cardiovascular coring apparatus may comprise a cutting member, a tissue retraction member, and a valve positioned inside the cutting member. A method for inserting a cutting member through a valve is disclosed. A method for inserting a tissue retraction member through a valve in a cutting member is also disclosed.

### BACKGROUND

Aortic valve replacement is a cardiac surgery procedure that replaces a patient's aortic valve with a prosthetic valve. Aortic valve replacement typically requires open heart surgery, which may be risky and/or impractical for many patients. Aortic valve replacement may not be an option for patients with aortic stenosis, left ventricular outflow obstruction, a heavily calcified ascending aorta, a heavily calcified aortic root, and/or other high risk medical conditions. For example, patients with conditions that preclude a median sternotomy may not be candidates for an aortic valve replacement operation.

Apical aortic conduits may provide a less invasive alternative to aortic valve replacement. An apical aortic conduit may be connected between the apex of the heart and the aorta in a procedure similar to a coronary artery bypass graft. Apical aortic conduits may improve blood flow between the heart and the aorta by bypassing a diseased or malfunctioning aortic valve. Patients who are not eligible for aortic valve replacement may be treated by using an apical aortic conduit to bypass the valve. For example, apical aortic conduits may be used in pediatric patients. The native valve may be left in place in pediatric patients to eliminate the need for periodic valve replacements as the patient grows. Thus, the apical aortic conduit may maintain the maximum possible function of the native valve while bypassing the restricted flow to lessen stress on the heart and allow more blood flow to the body. In other words, the apical aortic conduit may bypass the native valve to allow for extra flow to the aorta while still allowing the maximum flow that the native valve can physiologically handle.

Traditional apical aortic conduits may fail or malfunction for various reasons. For example, the conduit material used in an apical aortic conduit may become blocked as a result of kinking. Traditional conduits may also become occluded and obstruct apical flow. Also, apical aortic conduits are typically sutured to the heart and the aorta, and the suturing may cause aneurisms at or near the attachment site. Apical aortic conduits may also cause gastrointestinal complications such as dysphagia and gastric erosion. Furthermore, implanting an apical aortic conduit on a beating heart may result in significant blood loss from the patient.

### SUMMARY

In certain embodiments, a system may comprise a tube with a first end dimensioned to be positioned against a cardiovascular organ. The system may comprise a cutting member adapted to cut an opening in the cardiovascular organ. The system may also comprise a first valve positioned inside the tube. The first valve may be adapted to open to allow the cutting member to pass through the first valve to the cardiovascular organ. The first valve may also be adapted to close after the cutting member is retracted through the first valve.

According to at least one embodiment, the first valve may be adapted to seal the cutting member to the tube when the cutting member is positioned within the tube. In various embodiments, the system may comprise a cardiovascular coring device. The cardiovascular coring device may comprise the cutting member, and the cutting member may be adapted to cut the opening in the cardiovascular organ by coring a cylindrical section of cardiovascular organ tissue out of the cardiovascular organ. The cardiovascular coring device may also comprise a tissue retraction member adapted to remove the cylindrical section of cardiovascular organ tissue from the cardiovascular organ.

According to various embodiments, the tissue retraction member may comprise a corkscrew anchor adapted to twist into cardiovascular organ tissue. In some embodiments, the corkscrew anchor may be adapted to rotate in a first direction and the cutting member may be adapted to rotate in a second direction, with the first direction being opposite the second direction. In some embodiments, the tissue retraction member may comprise barbs.

According to at least one embodiment, the system may comprise a second valve positioned inside the cutting member. The second valve may be adapted to open to allow the tissue retraction member to pass through the second valve to the coring site. The second valve may be adapted to close after the tissue retraction member is retracted through the second valve. In some embodiments, the second valve may be adapted to seal the tissue retraction member to the cutting member while the tissue retraction member is positioned within the cutting member. According to various embodiments, the valve may comprise an expandable balloon. In some embodiments, the valve may comprise a one-way valve.

In certain embodiments, an apparatus may comprise a cutting member. The cutting member may be adapted to core an opening in a cardiovascular organ by cutting a section of tissue out of the cardiovascular organ. The apparatus may comprise a tissue retraction member. The tissue retraction member may be adapted to remove the section of tissue from the cardiovascular organ. The apparatus may also comprise a valve positioned inside the cutting member. The valve may be adapted to open to allow the tissue retraction member to pass through the valve to be inserted into the cardiovascular organ. The valve may also be adapted to close after the tissue retraction member is retracted through the valve.

According to various embodiments, the valve may be adapted to seal the tissue retraction member to the cutting member when the tissue retraction member is positioned within the cutting member. In some embodiments, the coring member may comprise a cylindrical tube. The cylindrical tube may comprise a handle at a proximal end and a cutting blade at a distal end. In at least one embodiment, the tissue retraction member may comprise a corkscrew anchor adapted to twist into cardiovascular organ tissue. According to some embodiments, the corkscrew anchor may be adapted to rotate in a first direction and the cutting member may be adapted to rotate in a second direction, with the first direction being opposite the second direction. According to various embodiments, the tissue retraction member may comprise barbs.

In certain embodiments, a method may comprise positioning a first end of a tube against a cardiovascular organ. The tube may comprise a first valve. The method may further comprise inserting a cutting member into the tube. The method may also comprise opening the first valve to allow the cutting member to pass through the valve to the cardiovascular organ. The method may comprise cutting a first section of tissue out of the cardiovascular organ with the cutting member. The method may comprise retracting the cutting member from the tube and closing the valve after the cutting member is retracted through the valve.

According to some embodiments, the method may further comprise anchoring a tissue retraction member in the first section of tissue of the cardiovascular organ. The method may also comprise retracting the first section of tissue out of the cardiovascular organ by retracting the tissue retraction member. According to various embodiments, the method may comprise inserting the tissue retraction member into the cutting member. The method may comprise opening a second valve to allow the tissue retraction member to pass through the second valve to the cardiovascular organ. The second valve may be positioned inside the cutting member to seal the cutting member to the tissue retraction member. According to various embodiments, the tissue retraction member may comprise a corkscrew anchor adapted to twist into cardiovascular organ tissue. In at least one embodiment, the tissue retraction member may comprise barbs. According to various embodiments, the first valve may seal the cutting member to the tube.

In certain embodiments, a system may comprise a tube with a first end dimensioned to be positioned against a coring site of a cardiovascular organ. The system may also comprise a cardiovascular coring device adapted to core an opening in the cardiovascular organ. The cardiovascular coring device may comprise a cutting member. The cutting member may be adapted to core the opening in the cardiovascular organ by cutting out a section of cardiovascular organ tissue. The coring device may also comprise a tissue retraction member. The tissue retraction member may be adapted to remove the section of cardiovascular organ tissue from the cardiovascular organ.

The system may further comprise a first valve positioned inside the tube. The first valve may be adapted to open to allow the cardiovascular coring device to pass through the first valve to the coring site. The first valve may be adapted to close after the cardiovascular coring device is retracted through the first valve. The first valve may also be adapted to seal the cardiovascular coring device to the tube when the cardiovascular coring device is positioned within the tube.

The system may comprise a second valve positioned inside the cutting member. The second valve may be adapted to open to allow the tissue retraction member to pass through the second valve to the coring site. The second valve may also be adapted to close after the tissue retraction member is retracted through the second valve. In various embodiments, the tissue retraction member may comprise a corkscrew anchor adapted to twist into cardiovascular organ tissue. According to at least one embodiment, the corkscrew anchor may be adapted to rotate in a first direction and the cutting member may be adapted to rotate in a second direction, with the first direction being opposite the second the direction.

In certain embodiments, a method may comprise positioning a cutting member against a cardiovascular organ. The cutting member may comprise a valve. The method may also comprise opening the valve to allow a tissue retraction member to pass through the valve to the cardiovascular organ. The method may comprise anchoring the tissue retraction member in a first section of tissue of the cardiovascular organ and cutting the first section of tissue out of the cardiovascular organ with the cutting member. The method may further comprise retracting the first section of tissue out of the cardiovascular organ by retracting the tissue retraction member and closing the valve after the tissue retraction member is retracted through the valve. In at least one embodiment, the tissue retraction member may comprise a corkscrew anchor adapted to twist into cardiovascular organ tissue. According to various embodiments, the cutting member may comprise a tapered cutting blade.

Features from any of the above-mentioned embodiments may be used in combination with one another in accordance with the general principles described herein. These and other embodiments, features, and advantages will be more fully understood upon reading the following detailed description in conjunction with the accompanying drawings and claims.

### BRIEF DESCRIPTION OF THE DRAWINGS

The accompanying drawings illustrate a number of exemplary embodiments and are part of the specification. Together with the following description, these drawings demonstrate and explain various principles of the instant disclosure.

FIG. 1 is a perspective view of a heart, an aorta, and an aorta measuring device according to certain embodiments.

FIG. 2 is a perspective view of an exemplary tube attached to an aorta according to certain embodiments.

FIG. 3 is a perspective view of an exemplary cardiovascular coring device being inserted into the tube illustrated in FIG. 2.

FIG. 4 is a perspective view of the cardiovascular coring device illustrated in FIG. 3.

FIG. 5 is another perspective view of the cardiovascular coring device illustrated in FIG. 3.

FIG. 6 is a perspective view of a valve sealing the tube illustrated in FIG. 5 against blood flowing out of an opening in an aorta.

FIG. 7 is a perspective view of inserting a cardiovascular conduit section into the tube shown in FIG. 6.

FIG. 8 is a perspective view of attaching a connector of the cardiovascular conduit system illustrated in FIG. 7 to an aorta.

FIG. 9 is a perspective view of an exemplary cardiovascular conduit system according to certain embodiments.

FIG. 10 is a perspective view of the cardiovascular conduit system illustrated in FIG. 9.

FIG. 11 is another perspective view of the cardiovascular conduit system illustrated in FIG. 9.

FIG. 12 is a perspective view of an exemplary cardiovascular coring device according to certain embodiments.

FIG. 13 is a perspective view of the exemplary cardiovascular coring device illustrated in FIG. 12.

FIG. 14 is a cross-sectional view of the exemplary cardiovascular coring device illustrated in FIG. 12.

FIG. 15 is another cross-sectional view of the exemplary cardiovascular coring device illustrated in FIG. 12.

FIG. 16 is a perspective view of an exemplary valve according to certain embodiments.

FIG. 17 is a perspective view of the valve illustrated in FIG. 16 in a closed position.

FIG. 18 is a perspective view of an exemplary cardiovascular coring device according to certain embodiments.

FIG. 19 is a perspective view of the exemplary cardiovascular coring device illustrated in FIG. 18.

FIG. 20 is a perspective view of an exemplary valve according to certain embodiments.

FIG. 21 is a perspective view of inserting a cardiovascular coring device through the valve illustrated in FIG. 20.

FIG. 22 is a perspective view of an exemplary cardiovascular coring device according to certain embodiments.

FIG. 23 is a perspective view of the cardiovascular coring device illustrated in FIG. 22.

FIG. 24 is another perspective view of the cardiovascular coring device illustrated in FIG. 22.

FIG. 25 is a perspective view of an exemplary cutting device according to certain embodiments.

FIG. 26 is a perspective view of an exemplary cutting device coring an opening in an aorta.

FIG. 27 is a perspective view of the exemplary cutting device illustrated in FIG. 26.

FIG. 28 is a perspective view of a cardiovascular conduit section being inserted into the cutting device illustrated in FIG. 26.

FIG. 29 is a perspective view of the cardiovascular conduit section illustrated in FIG. 28 being attached to an aorta.

FIG. 30 is a perspective view of the cardiovascular conduit section illustrated in FIG. 28 attached to an aorta.

Throughout the drawings, identical reference characters and descriptions indicate similar, but not necessarily identical, elements. While the exemplary embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

A physician may implant a cardiovascular conduit system to circumvent a restriction in blood flow. For example, a physician may use a cardiovascular conduit system to bypass an aortic valve in a patient with aortic valve stenosis. Similarly, a cardiovascular conduit system may be used to bypass a pulmonary valve in a patient with pulmonary valve stenosis. Physicians may also use cardiovascular conduit systems to address various other problems and diseases in a patient's cardiovascular system.

Cardiovascular conduit systems may provide various advantages over prior systems. Physicians may implant a cardiovascular conduit system on a beating heart. Procedures performed on a beating heart may be referred to as off-pump procedures, and off-pump procedures may be less invasive than on-pump procedures (i.e., procedures that require cardiopulmonary bypass). In some embodiments, cardiovascular conduit systems may be used with traditional surgical techniques (e.g., on-pump procedures). In traditional surgical techniques, cardiovascular conduit systems may provide various advantages, such as reduced pump time and smaller incisions. Connectors in a cardio-vascular conduit system may be designed to reduce the risk of aneurisms at the attachment site. The conduit in a cardiovascular conduit system may be kink and occlusion resistant. Cardiovascular conduit systems may also reduce the risk of gastrointestinal complications. Cardiovascular conduit systems may be implanted quickly and minimize patient blood loss. The following disclosure presents numerous other features and advantages of cardiovascular conduit systems.

The process of implanting a cardiovascular conduit system in a patient may involve a variety of steps. FIGS. 1-11 illustrate an exemplary process for implanting a cardiovascular conduit system between an apex of a heart and an aorta. The first step in implanting a cardiovascular conduit system may be measuring the size of a patient's aorta. A physician may determine the size of the patient's aorta to determine the appropriate sizes for the coring device and aortic connector that will be used in the procedure.

FIG. 1 illustrates an aorta measuring device 100 measuring a circumference of an aorta 14. Aorta measuring device 100 may include a handle 110, an extension 120, and a circular measuring member 140. A physician may position measuring member 140 around aorta 14. The physician may then tighten measuring member 140 until it is snug around aorta 14 and capable of measuring the circumference of aorta 14. The physician may then take a measurement from measuring member 140. Various examples of aorta measuring devices are illustrated and described in U.S. patent application Ser. No. 12/340,382, filed on 19 Dec. 2008, and entitled “Apparatus and Method for Measuring Blood Vessels,” the disclosure of which is incorporated in its entirety by this reference.

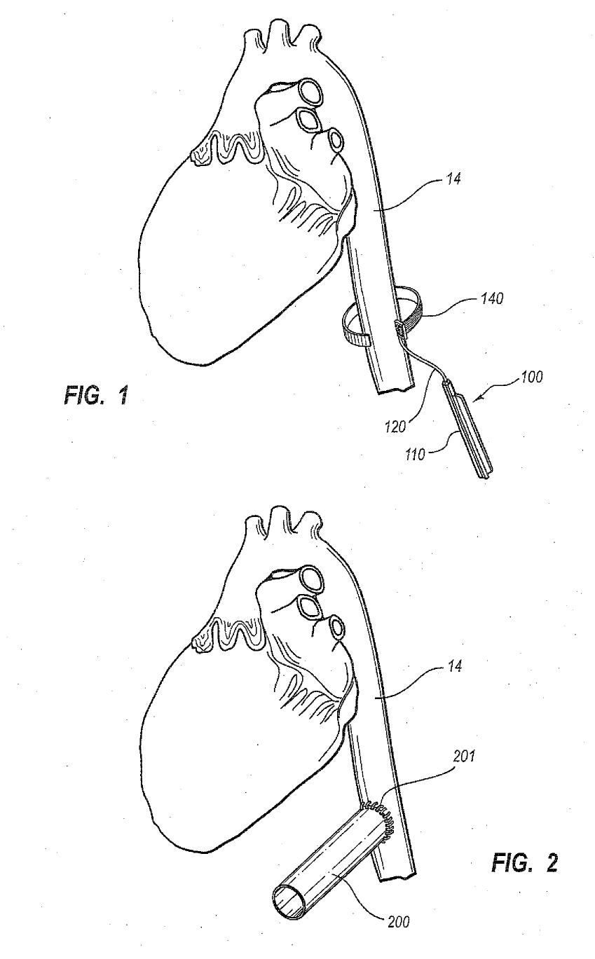

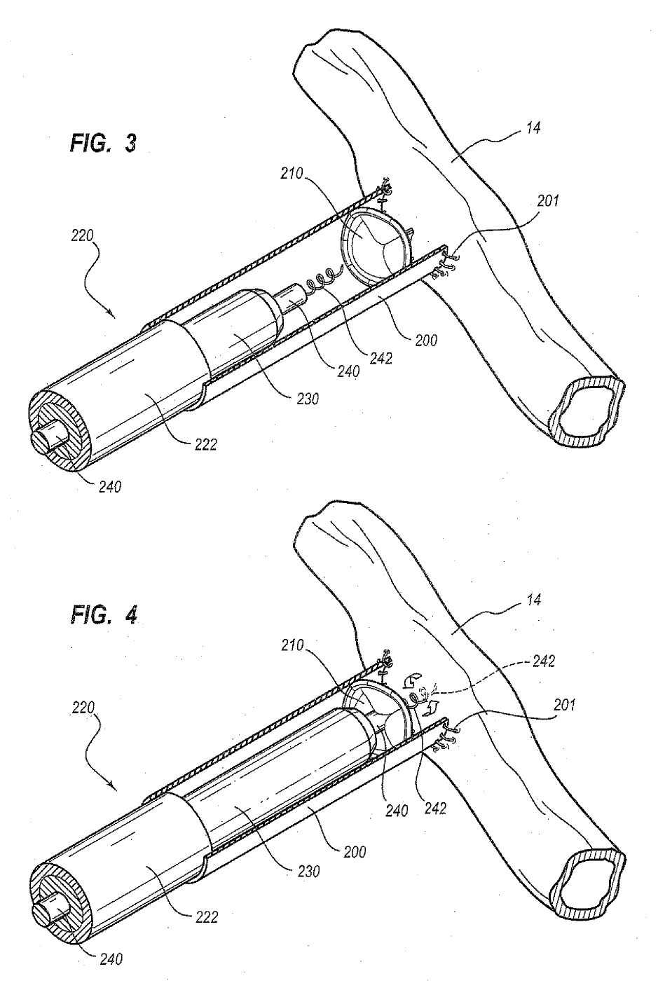

After determining the size of a patient's aorta, the physician may select the appropriately sized connector, conduit, valve, coring device, tube, and/or other tools for implanting the cardiovascular conduit system. FIG. 2 shows an end of a tube 200 attached to aorta 14. As shown in FIGS. 3-8, tube 200 may provide a sealed interface with aorta 14 during various steps in the process of implanting a cardiovascular conduit system. Tube 200 may be any suitable size and/or shape. As shown in FIG. 2, tube 200 may be cylindrical. In other embodiments, tube 200 may have a rectangular shape, square shape, triangular shape, or any other suitable shape. Tube 200 may be any suitable length and may be made of any suitable material (e.g., metal, plastic, etc.). Tube 200 may be any suitable type of duct, conduit, pipe, channel, or other enclosure designed to provide a sealed interface between an aorta and various cardiovascular conduit system parts and tools.

As shown in FIG. 2, tube 200 may be sutured to aorta 14 by sutures 201. Sutures 201 may hold tube 200 in place and may help prevent blood leakage at the interface between aorta 14 and tube 200. Tube 200 may be secured to aorta 14 using any suitable attachment mechanism in addition to or instead of sutures. For example, tube 200 may be secured to aorta 14 using a clamp that wraps around aorta 14. In other embodiments, a physician may press tube 200 against aorta 14 without using any additional attachment mechanism.

A physician may insert a coring device into tube 200 after attaching tube 200 to aorta 14. FIG. 3 shows a cross-sectional view of tube 200. FIG. 3 also illustrates a cardiovascular coring device 220 being inserted into tube 200. Cardiovascular coring device 220 may include a handle 222, a cutting member 230, a tissue retraction member 240, and a corkscrew anchor 242. Corkscrew anchor 242 may extend from tissue retraction member 240. Tissue retraction member 240 may extend through cutting member 230 and handle 222.

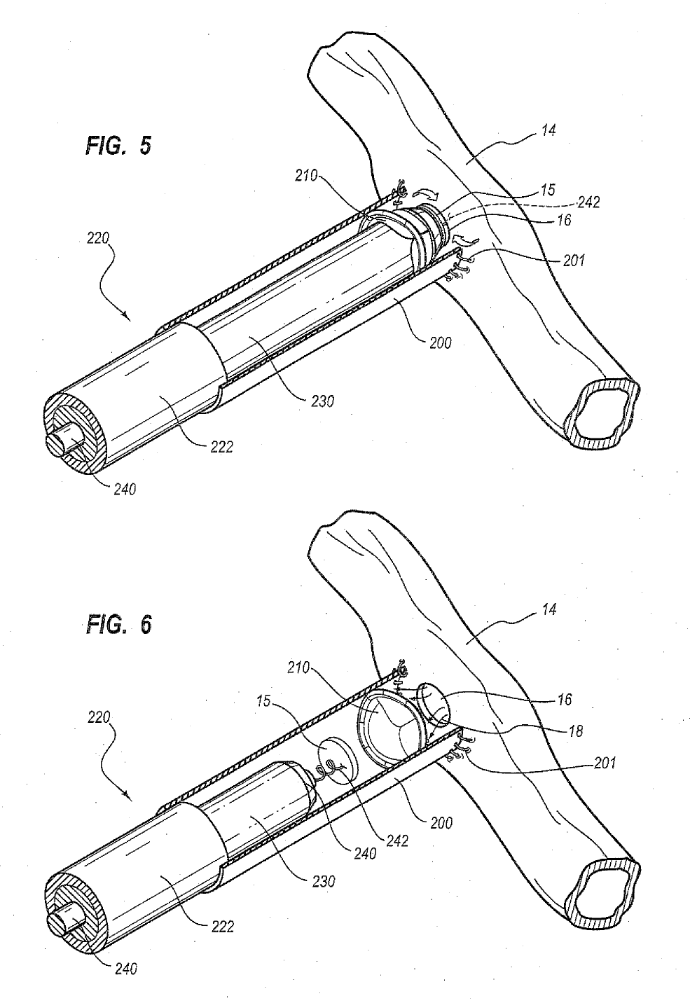

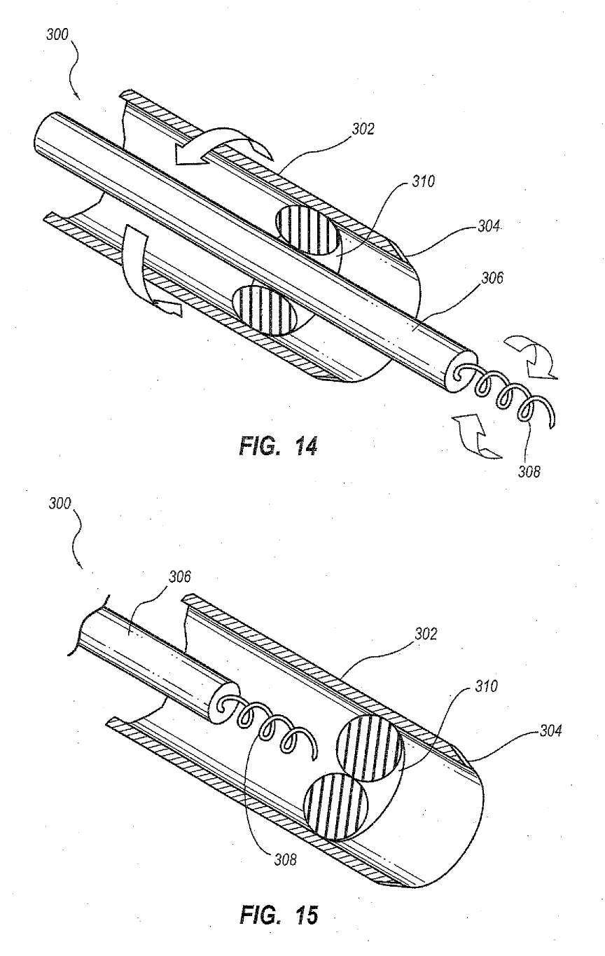

A valve 210 may be positioned within tube 200. Valve 210 may be attached to tube 200. In other embodiments, valve 210 may be formed as part of tube 200. Valve 210 may be a one-way valve that allows cardiovascular coring device 220 to pass through but blocks the flow of blood out of the opening cut in aorta 14. Valve 210 may also seal cutting member 230 to tube 200 (as shown in FIG. 5) to prevent blood from leaking between tube 200 and cutting member 230. FIGS. 14-25 illustrate various examples of valves that may be used in a delivery tube.

FIG. 4 illustrates corkscrew anchor 242 being rotated into artery 14. Corkscrew anchor 242 may be secured to the section of tissue that will be removed from artery 14. Corkscrew anchor 242 may prevent the section of tissue from entering the blood stream in aorta 14 or causing other problems in the implant procedure. Various other types of anchors may be secured to cardiovascular organ tissue, as will be discussed in the disclosure corresponding to FIGS. 12-24.

FIG. 5 shows cardiovascular coring device 220 cutting an opening in aorta 14. Valve 210 may seal cutting member 230 to tube 200 while cutting member 230 cuts the opening in aorta 14. Cutting member 230 may rotate to cut the opening in aorta 14. In some embodiments, cutting member 230 may be directly connected to handle 222, and a physician may rotate cutting member 230 by rotating handle 222. In other embodiments, cutting member 230 may be rotated by an electric motor or any other suitable rotating mechanism.

According to various embodiments, cutting members may be any cutting devices suitable for cutting a cardiovascular organ. A cutting member may be a mechanical coring device, as illustrated in FIG. 5. A cutting member may also be, for example, a laser scalpel, a high-frequency ultra-sound device, or any other suitable type of cutting device. Cutting members may be standalone devices. In other embodiments, a cutting member may be incorporated into a cardiovascular coring device or any other suitable device.

FIG. 6 shows artery 15 with an opening 16 that was cut open by cutting member 230. Corkscrew anchor 242 may be attached to a section of tissue 15 of aorta 14 that was cut out by cutting member 230. A physician may retract cardiovascular coring device 220 to pull tissue 15 away from artery 14, as shown in FIG. 6. In some embodiments, tissue retraction member 240 may be retracted into cutting member 240 before cutting member 240 is retracted through valve 210. In various embodiments, tissue retraction member 240 may be completely retracted out of cutting member 230 and handle 240. Valve 210 may close after cutting member 240 is retracted through valve 210, thereby preventing blood 18 from flowing out of tube 200.

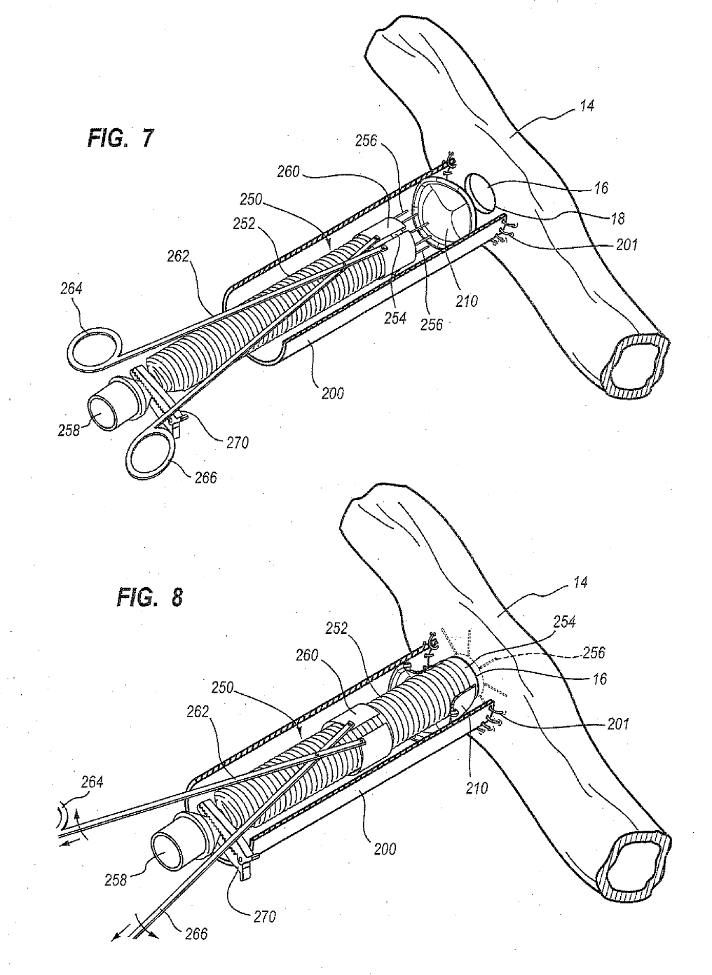

FIG. 7 shows a cardiovascular conduit system 250 inserted into tube 200. Cardiovascular conduit system 250 may be inserted into tube 200 after cardiovascular coring device 220 is retracted from tube 200. Cardiovascular conduit section 250 may include a conduit 252, a connector 258, and a connector 254. Various examples of cardiovascular conduits and connectors are shown and discussed in U.S. patent application Ser. No. 12/340,280, filed on 19 Dec. 2008, and entitled “Systems, Apparatuses, and Methods for Cardiovascular Conduits and Connectors,” the disclosure of which is incorporated in its entirety in this reference.

Connector 254 may include expandable members 256. A retractable retaining member 260 may hold expandable members 256 in a delivery position while connector 254 is being implanted into aorta 14. Retractable retaining member 260 may be attached to handles 264 and 266 to allow a physician to control retractable retaining member 260.

A distal end of cardiovascular conduit section 250 may be sealed with a clamp 270. Clamp 270 may prevent blood from flowing out of cardiovascular conduit section 250 through connector 258 after cardiovascular conduit section 250 is attached to aorta 14. Clamp 270 may be any suitable size, shape, and/or configuration.

As shown in FIG. 8, valve 210 may open to allow cardiovascular conduit section 250 pass through valve 210. As with cardiovascular coring device 220, cardiovascular conduit section 250 may be sealed to tube 200 by valve 210. Thus, valve 210 may prevent blood from leaking while cardiovascular conduit section 250 is being secured to aorta 14.

Connector 254 may pass through valve 210 and be partially inserted into opening 16 of aorta 14 such that expandable members 256 extend into aorta 14. A physician may then retract retaining member 260 to allow expandable members 256 to deploy and secure cardiovascular conduit system 250 to aorta 14, as shown in FIG. 8. After cardiovascular conduit section 250 is implanted in aorta 14, tube 200 may be removed from aorta 14. In embodiments where tube 200 was sutured to aorta 14, the sutures, such as sutures 201, may be removed and tube 200 may be retracted from the implant site on aorta 14.

A physician may use a procedure similar to or the same as the procedure for implanting conduit section 250 in aorta 14 for implanting a cardiovascular conduit section in an apex of the heart at the left ventricle. For example, a tube may be attached to an apex of the heart. Then, a cutting member may be inserted through a valve in the tube to cut out a section of the apex of the heart. After the section of the heart and the cutting member are removed from the tube, a cardiovascular conduit section may be inserted through the tube and attached to the apex of the heart. This procedure (or similar procedures) for implanting cardiovascular conduit sections may be performed on the left ventricle of the heart, the right ventricle of the heart, the pulmonary artery, or any other blood vessel or cardiovascular organ.

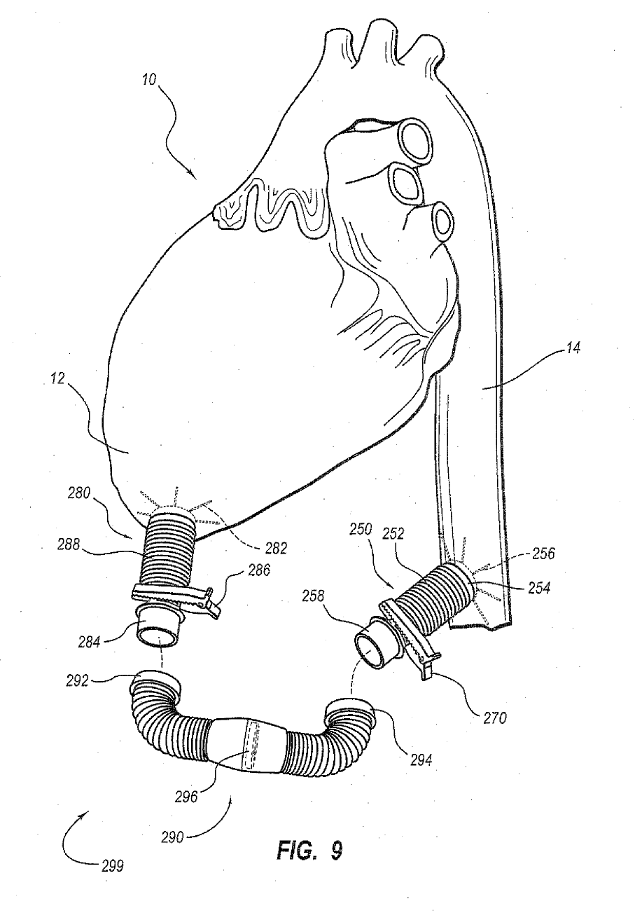

FIG. 9 illustrates a cardiovascular conduit system 299. Cardiovascular conduit system 299 may include a cardiovascular conduit section 280 attached to a left ventricle 12 of heart 10 at an apex of heart 10. Cardiovascular conduit section 280 may include a connector 282, a connector 284, and a conduit 288. Connector 282 may be attached to left ventricle 12, and conduit 288 may be sealed against blood leakage between connectors 282 and 284 by clamp 286.

Cardiovascular conduit system 299 may also include cardiovascular conduit section 250, which includes connector 254, connector 258, and conduit 252. As previously noted, connector 254 may be attached to aorta 14. FIG. 9 also shows that cardiovascular conduit system 299 may include a cardiovascular conduit section 290. Cardiovascular conduit section 290 may include a connector 292, a connector 294, and a valve 296. Various examples of valves and valve housings are illustrated and described in U.S. patent application Ser. No. 12/340,189, filed on 19 Dec. 2008, and entitled “Cardiovascular Valve and Valve Housing Apparatuses and Systems,” the disclosure of which is incorporated in its entirety by this reference. Connector 292 may be dimensioned to attach to connector 284, and connector 294 may be dimensioned to attach to connector 258.

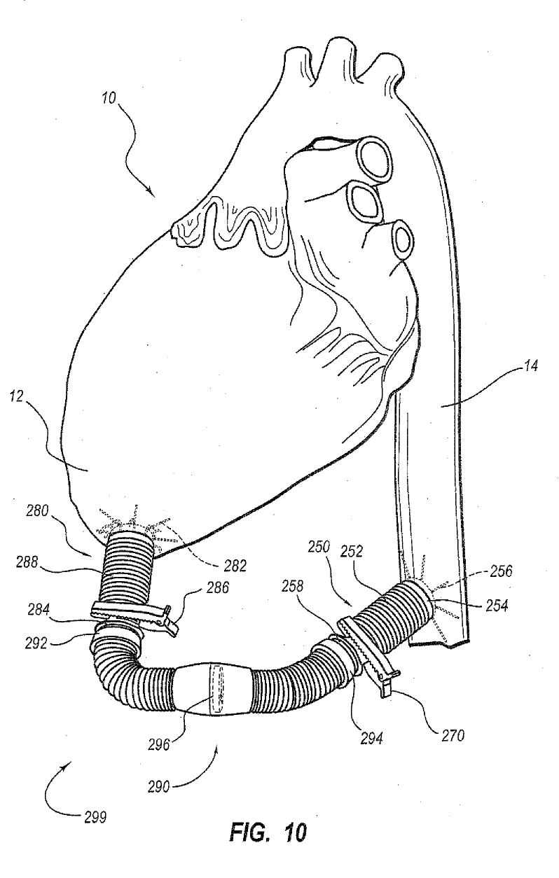

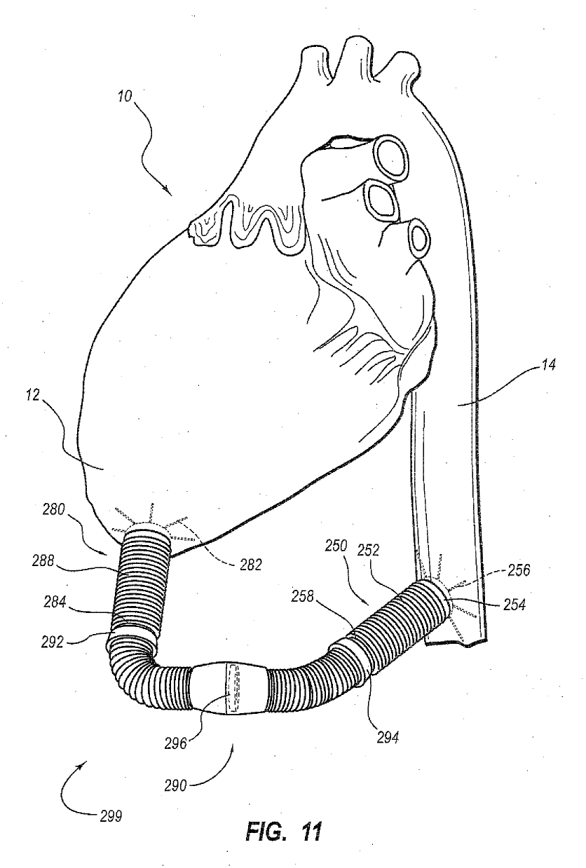

FIG. 10 shows cardiovascular conduit section 290 attached between cardiovascular conduit sections 250 and 280. Connector 292 may be attached to connector 284 to join cardiovascular conduit section 280 with cardiovascular conduit section 290. Similarly, connector 294 may be attached to connector 258 to join cardiovascular conduit sections 250 and 290. After cardiovascular conduit system 299 is assembled, clamps 270 and 286 may be removed to allow blood to begin to flow between left ventricle 12 and aorta 14. FIG. 11 shows cardiovascular conduit system 299 with clamps 270 and 286 removed. After clamps 270 and 286 are removed, blood may flow from left ventricle 12 to aorta 14 through valve 296.

Cardiovascular conduit systems, such as cardiovascular conduit system 299, may be attached between various cardiovascular organs. A cardiovascular organ may be any organ in a cardiovascular system. Cardiovascular organs include the heart and all the blood vessels (e.g., arteries and veins) in the cardiovascular system. Thus, the aorta and the pulmonary artery may be referred to as cardiovascular organs. According to some embodiments, blood vessels may also be referred to as vascular organs.

Various different types of cutting devices and valves may be used in the process of implanting a cardiovascular conduit system in a patient. According to some embodiments, valves may also be referred to as occluding devices. FIGS. 12-25 show various examples of valves and cutting devices.

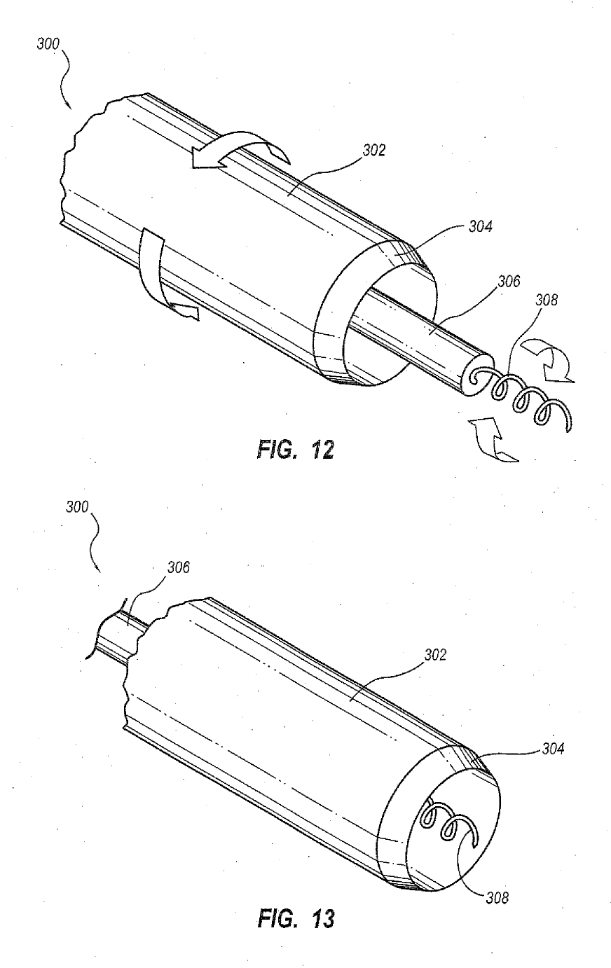

FIGS. 12 and 13 illustrate a cardiovascular coring device 300. Cardiovascular coring device 300 may include a cutting member 302 and a tissue retraction member 306. Cutting member 302 may include a blade 304. Cutting member 302 may be made of any suitable material. In some embodiments, cutting member 302 may be made of a biocompatible metal, such as stainless steel. As previously noted, cutting member 302 may be attached to a handle or formed as part of a handle.

Tissue retraction member 306 may also be made of any suitable material, such as a biocompatible metal. Corkscrew anchor 308 may extend from a proximal end of tissue retraction member 306. A physician may twist corkscrew anchor 308 to secure corkscrew anchor 308 to the tissue of a cardiovascular organ. After corkscrew anchor 308 is secured to the cardiovascular organ, cutting member 302 may core an opening in the cardiovascular organ. As indicated by the arrows in FIG. 12, cutting member 302 may be rotated in the opposite direction as corkscrew anchor 308 to ensure that the tissue held by corkscrew anchor 308 is not accidentally released. Then, tissue retraction member 306 may be retracted, as illustrated in FIG. 13. As tissue retraction member 306 is retracted, tissue retraction member 306 may remove a section of a cardiovascular organ cut out by cutting member 302.

FIGS. 14 and 15 show a cross-section of cutting member 302 with tissue retraction member 306 being retracted through cutting member 302. Cutting member 302 may include a valve 310 to prevent blood leakage between cutting member 302 and tissue retraction member 306. Valve 310 may have a toroidal shape and may be an expandable balloon type valve. Valve 310 may also be any other suitable type of valve.

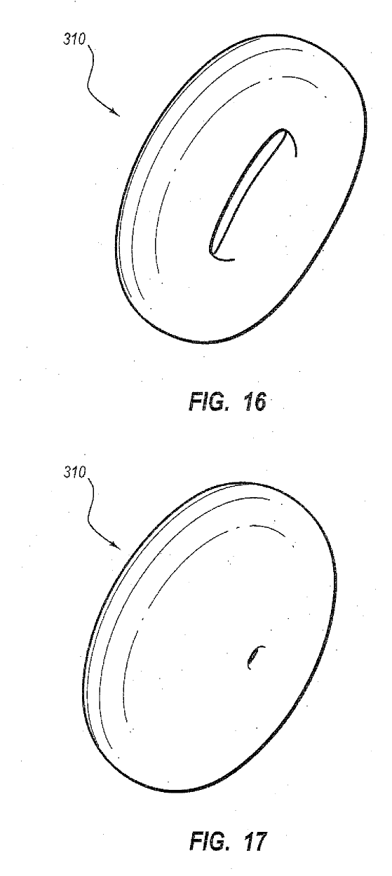

As shown in FIG. 15, after tissue retraction member 306 is retracted through valve 310, valve 310 may close to prevent blood from exiting through cutting member 302. Thus, in some embodiments, tissue retraction member 306 may completely remove a section of cardiovascular organ tissue through valve 310 with minimal blood loss. FIGS. 16 and 17 are perspective views of balloon-expandable valve 310. Balloon-expandable valve 310 may have a toroidal shape. Balloon expandable valves may also have any other suitable shape, according to some embodiments. As shown in FIG. 16, in an open position, valve 310 may have a central opening 312 through which a tissue retraction member may pass. FIG. 17 shows that valve 310 may close opening 312 to completely seal itself against blood flow.

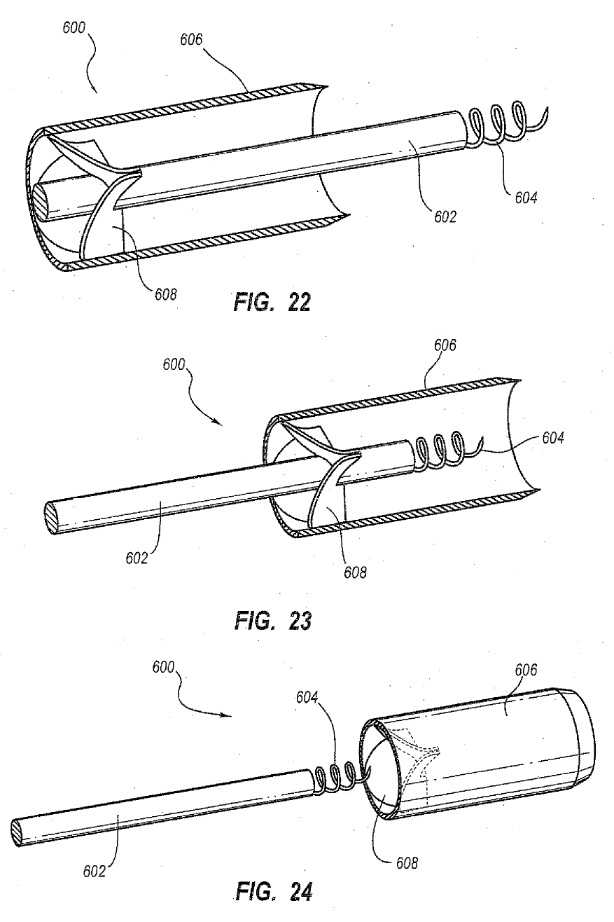

Valve 310 may be any suitable valve. For example, valve 310 may be a one-way valve, such as the valves illustrated in FIGS. 22-25. In some embodiments, tissue retraction member 306 may not be removable from cutting member 302. In such embodiments, cutting member 302 may not include a valve. For example, tissue retraction member 306 may be sealed to cutting member 302 using a washer or any other suitable sealing device.

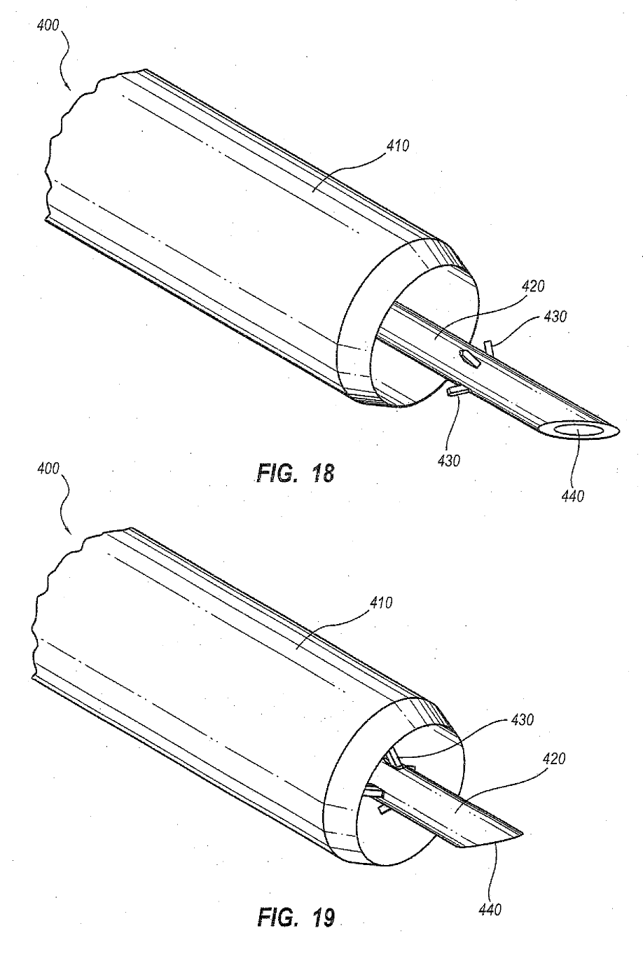

FIGS. 18 and 19 show a cardiovascular coring device 400. Cardiovascular coring device 400 may include a cutting member 410 and a tissue retraction member 420. Tissue retraction member 420 may include barbs 430. Barbs 430 may also be referred to as hooks or spires. Tissue retraction member 420 may have a sharp tip 440 to allow tissue retraction member 420 to puncture a cardiovascular organ at a coring site. After tissue retraction member 420 punctures the cardiovascular organ, tissue retraction member 420 may be pressed into the cardiovascular organ until barbs 430 are inside the cardiovascular organ. After cutting member 410 cores an opening in the cardiovascular organ, tissue retraction member 420 may be retracted into cutting member 410, as shown in FIG. 19. Barbs 430 may hold the piece of cardiovascular tissue cored by cutting member 410 as tissue retraction member 420 is retracted into cutting member 410. Thus, barbs 430 may allow tissue retraction member 420 to pull the piece of cored tissue from the cardiovascular organ.

According to some embodiments, tissue retraction member 420 may be a tube with a needle-shaped end for piercing a cardiovascular organ. Barbs 430 may be laser cut from the tube. In some embodiments, barbs 430 may be fixed at any suitable angle and may then be annealed into place. According to various embodiments, tissue retraction member 420 may not be hollow all the way through or may not be hollow at all.

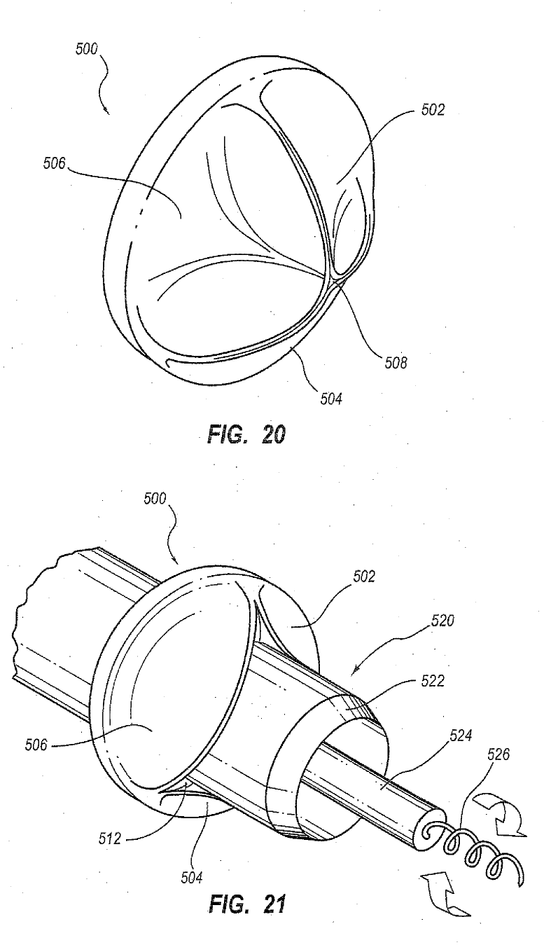

FIG. 20 is a perspective view of a valve 500 for use with a cardiovascular coring device. Valve 500 may be used inside a cutting member or inside a tube, as shown in FIG. 3. Valve 500 may be made of any suitable material, such as a polymer. Valve 500 may include a section 502, a section 504, and a section 506. Valve 500 may be a one-way valve that prevents fluid from exiting the valve when it is closed. Valve 500 may open to expose an opening 508.

FIG. 21 shows a cardiovascular coring device 520 being inserted through valve 500. As shown, sections 502, 504, and 506 of valve 500 may open to allow cardiovascular coring device 520 to pass through. Valve 500 may also include sealing sections 510 and 512 that press against the outside of cutting member 522 of cardiovascular coring device 520 to seal cutting member 522 to valve 500 and prevent blood from leaking between cutting member 522 and valve 500.

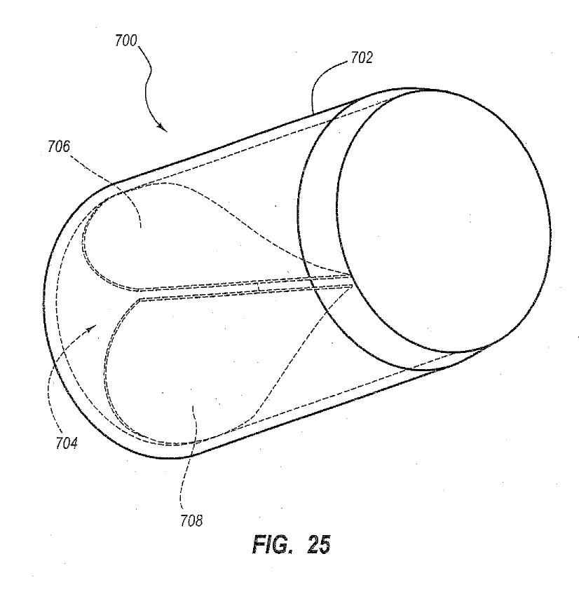

FIG. 22 illustrates a cardiovascular coring device with a valve 608, a tissue retraction member 602, and a cutting member 606. Tissue retraction member 602 may include an anchor 604. Tissue retraction member 602 may be any suitable size, shape, and/or configuration. FIGS. 23 and 24 show tissue retraction member 602 being retracted out of cutting member 606 through valve 608. As shown in FIGS. 22-24, valve 608 may be a three-section valve. According to various embodiments, valve 608 may be any suitable size, shape, and/or configuration. For example, FIG. 25 illustrates a cardiovascular coring device 700 with a two-section valve 704. Valve 704 may include sections 706 and 708 positioned within a cutting member 702.

Valves may be positioned at any suitable location within a cutting member. For example, as shown in FIG. 25, valve 704 may be positioned at a distal end (i.e., the end opposite the cutting blade) of cutting member 702. According to various embodiments, a valve may be positioned in the middle of the cutting member or near the cutting blade of the cutting member.

Various other shapes, sizes, and embodiments of valves may be implemented for use with cardiovascular coring devices. The valves and corresponding coring devices disclosed herein may allow a cardiovascular conduit system to be implanted quickly with minimal blood leakage. Thus, the coring devices and valves disclosed herein may improve the speed of implantation and minimize blood loss during implantation of a cardiovascular conduit system.

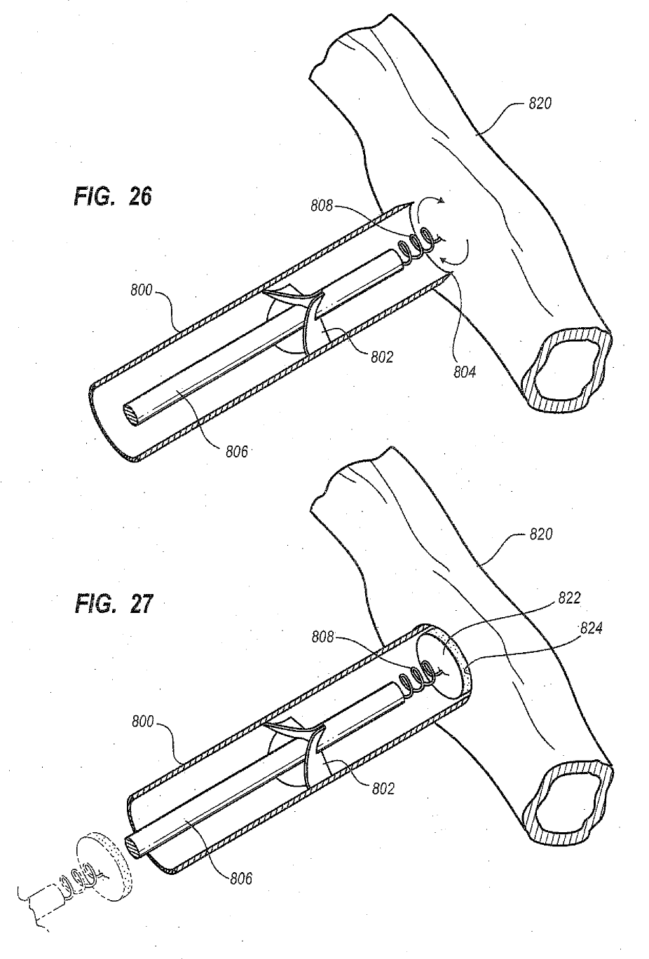

FIGS. 26-30 illustrate an embodiment of coring an opening in an aorta and attaching a cardiovascular conduit section to the opening in the aorta. FIG. 26 shows a cross-sectional view of a cutting member 800 with a blade 804 rotating to cut an opening in an aorta 820. A valve 802 may be positioned inside cutting member 800, and a tissue retraction member 806 may pass through valve 802. Tissue retraction member 806 may include an anchor 808 that is attached to aorta 820. FIG. 26 shows that a tube does not necessarily need to be attached to aorta 820 when coring an opening in aorta 820. Cutting member 800 may seal against the opening in aorta 820 to prevent blood from leaking around cutting member 800. In some embodiments, blade 804 may be tapered toward the inside of cutting member 800 so that the diameter of the opening cored by blade 804 is smaller than the diameter of cutting member 800. When cutting member 800 is pressed into the smaller opening, the opening may apply a radial force against cutting member 800 and help seal aorta 820 to cutting member 800 during the process of attaching a connector to aorta 820.

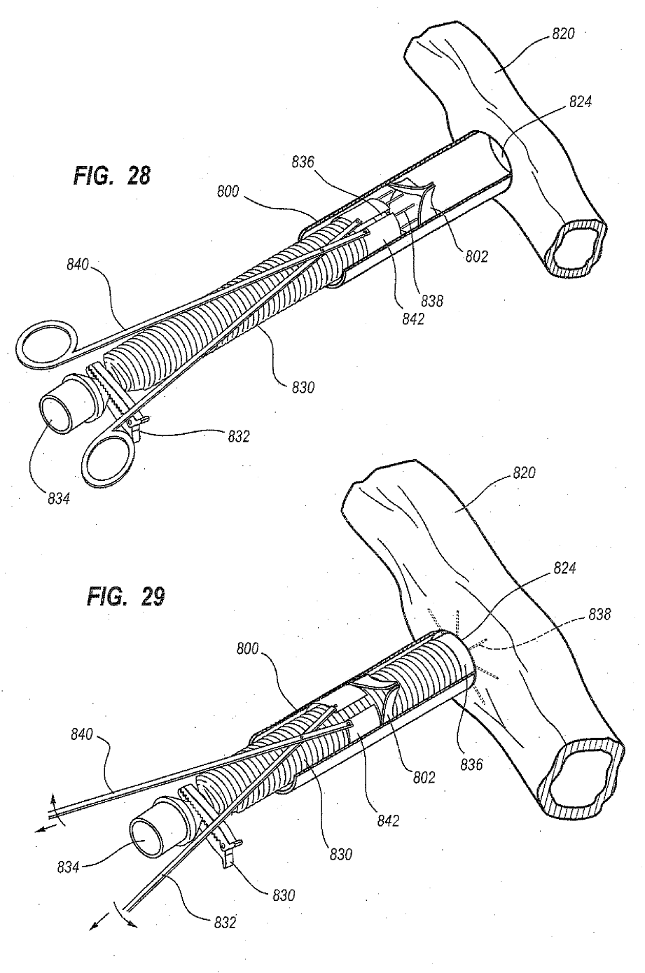

FIG. 27 shows cutting member 800 inserted into an opening 824 in aorta 820. Anchor 808 may be attached to a cored pieced 822 of aorta 820. The cored piece 822 of aorta 820 may be retracted through valve 802. Valve 802 may prevent blood from leaking out of cutting member 800 after the cored piece 822 of aorta 820 is removed from aorta 820. FIG. 28 shows that a cardiovascular conduit section 830 may be inserted into cutting member 800 after tissue retraction member 806 is removed from cutting member 800. Cardiovascular conduit section 830 may include a connector 834 and a connector 836. Connector 836 may include expandable members 838. A retractable retaining device 840 may include a retractable retaining member 842 that holds expandable members 838 in a delivery configuration. A distal end of cardiovascular conduit section 830 may be clamped by a clamp 832.

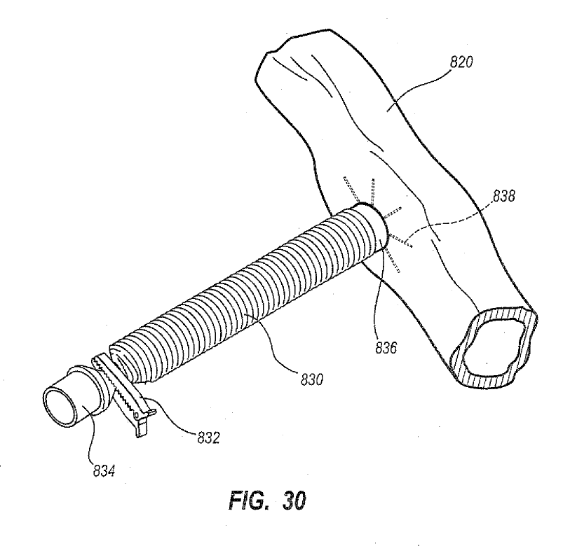

FIG. 29 shows that cardiovascular conduit section 830 may pass through valve 802, and connector 836 may be inserted into opening 824. Retractable retaining member 842 may be retracted to allow expandable members 838 to expand inside of aorta 820. Expandable members 838 may help secure connector 836 to aorta 820. After connector 836 is secured to aorta 820, cutting member 800 may be retracted, leaving cardiovascular conduit section 830 attached to aorta 820, as shown in FIG. 30. The cutting member and process illustrated in FIGS. 26-30 may also be used to secure a cardiovascular conduit section to other cardiovascular organs (e.g., a heart). The process and cutting member illustrated in FIGS. 26-30 may minimize trauma to the cardiovascular organ at the coring site. The features illustrated in FIGS. 26-30 may also allow a physician to quickly and efficiently implant a cardiovascular conduit system.

The preceding description has been provided to enable others skilled in the art to best utilize various aspects of the exemplary embodiments described herein. This exemplary description is not intended to be exhaustive or to be limited to any precise form disclosed. Many modifications and variations are possible without departing from the spirit and scope of the instant disclosure. It is desired that the embodiments described herein be considered in all respects illustrative and not restrictive and that reference be made to the appended claims and their equivalents for determining the scope of the instant disclosure.

Unless otherwise noted, the terms “a” or “an”, as used in the specification and claims, are to be construed as meaning “at least one of.” In addition, for ease of use, the words “including” and “having”, as used in the specification and claims, are interchangeable with and have the same meaning as the word “comprising.”

What is claimed is:

1. A system comprising:
a tube with a first end dimensioned to be positioned against a cardiovascular organ;
a cutting member adapted to cut an opening in the cardiovascular organ;
a first valve positioned inside the tube, the first valve being adapted to open to allow the cutting member to pass through the first valve to the cardiovascular organ, the first valve being adapted to close after the cutting device is retracted through the first valve.

2. The system of claim 1, wherein the first valve is adapted to seal the cutting member to the tube when the cutting member is positioned within the tube.

3. The system of claim 1, further comprising a cardiovascular coring device, the cardiovascular coring device comprising:
the cutting member, wherein the cutting member is adapted to cut the opening ii the cardiovascular organ by coring a cylindrical section of cardiovascular organ tissue out of the cardiovascular organ;
a tissue retraction member adapted to remove the cylindrical section of cardiovascular organ tissue from the cardiovascular organ.

4. The system of claim 3, wherein the tissue retraction member comprises a corkscrew anchor adapted to twist into cardiovascular organ tissue.

5. The system of claim 4, wherein the corkscrew anchor is adapted to rotate in a first direction and the cutting member is adapted to rotate in a second direction, the first direction being opposite the second direction.

6. The system of claim 3, wherein the tissue retraction member comprises barbs.

7. The system of claim 3, further comprising:
a second valve positioned inside the cutting member, the second valve being adapted to open to allow the tissue retraction member to pass through the second valve to the cardiovascular organ, the second valve being adapted to close after the tissue retraction member is retracted through the second valve.

8. The system of claim 7, wherein the second valve is adapted to seal the tissue retraction member to the cutting member while the tissue retraction member is positioned within the cutting member.

9. The system of claim 1, wherein the valve comprises an expandable balloon.

10. The system of claim 1, wherein the valve comprises a one-way valve.

11. An apparatus comprising:
a cutting member adapted to core an opening in a cardiovascular organ by cutting a section of cardiovascular organ tissue out of the cardiovascular organ;
a tissue retraction member adapted to remove the section of cardiovascular organ tissue from the cardiovascular organ;
a valve positioned inside the cutting member, the valve being adapted to open to allow the tissue retraction member to pass through the valve to be inserted into the cardiovascular organ, the valve being adapted to close after the tissue retraction member is retracted through the valve.

12. The apparatus of claim 11, wherein the valve is adapted to seal the tissue retraction member to the cutting member while the tissue retraction member is positioned within the cutting member.

13. The apparatus of claim 11, wherein the cutting member comprises a cylindrical tube.

14. The apparatus of claim 13, wherein the cylindrical tube comprises a handle at a proximal end and a cutting blade at a distal end.

15. The apparatus of claim 11, wherein the tissue retraction member comprises a corkscrew anchor adapted to twist into cardiovascular organ tissue.

16. The apparatus of claim 15, wherein the corkscrew anchor is adapted to rotate in a first direction and the cutting member is adapted to rotate in a second direction, the first direction being opposite the second direction.

17. The apparatus of claim 11, wherein the tissue retraction member comprises barbs.

18. A method comprising:
positioning a first end of a tube against a cardiovascular organ, the tube comprising a first valve;
inserting a cutting member into the tube;
opening the first valve to allow the cutting member to pass through the first valve to the cardiovascular organ;
cutting a first section of tissue out of the cardiovascular organ with the cutting member;
retracting the cutting member from the tube;
closing the first valve after the cutting member is retracted through the first valve.

19. The method of claim 18, further comprising:
anchoring a tissue retraction member in the first section of tissue of the cardiovascular organ;
retracting the first section of tissue out of the cardiovascular organ by retracting the tissue retraction member.

20. The method of claim 19, further comprising:
inserting the tissue retraction member into the cutting member;
opening a second valve to allow the tissue retraction member to pass through the second valve to the cardiovascular organ, the second valve being positioned inside the cutting member to seal the cutting member to the tissue retraction member.

21. The method of claim 19, wherein the tissue retraction member comprises a corkscrew anchor adapted to twist into cardiovascular organ tissue.

22. The method of claim 19, wherein the tissue retraction member comprises barbs.

23. The method of claim 18, wherein the first valve seals the cutting member to the tube.

24. A system comprising:
a tube with a first end dimensioned to be positioned against a coring site of a cardiovascular organ;
a cardiovascular coring device adapted to core an opening in the cardiovascular organ, the cardiovascular coring device comprising:
a cutting member adapted to core the opening in the cardiovascular organ by cutting out a section of cardiovascular organ tissue;
a tissue retraction member adapted to remove the section of cardiovascular organ tissue from the cardiovascular organ;
a first valve positioned inside the tube, the first valve being adapted to open to allow the cardiovascular coring device to pass through the first valve to the coring site, the first valve being adapted to close after the cardiovascular coring device is retracted through the first valve, the first valve being adapted to seal the cardiovascular coring device to the tube when the cardiovascular coring device is positioned within the tube;
a second valve positioned inside the cutting member, the second valve being adapted to open to allow the tissue retraction member to pass through the second valve to the coring site, the second valve being adapted to close after the tissue retraction member is retracted through the second valve.

25. The system of claim 24, wherein the tissue retraction member comprises a corkscrew anchor adapted to twist into cardiovascular organ tissue.

26. The system of claim 25, wherein the corkscrew anchor is adapted to rotate in a first direction and the cutting member is adapted to rotate in a second direction, the first direction being opposite the second direction.

27. A method comprising:
positioning a cutting member against a cardiovascular organ, the cutting member comprising a valve;
opening the valve to allow a tissue retraction member to pass through the valve to the cardiovascular organ;
anchoring the tissue retraction member in a first section of tissue of the cardiovascular organ;
cutting the first section of tissue out of the cardiovascular organ with the cutting member;
retracting the first section of tissue out of the cardiovascular organ by retracting the tissue retraction member;
closing the valve after the tissue retraction member is retracted through the valve.

28. The method of claim 27, wherein the tissue retraction member comprises a corkscrew anchor adapted to twist into cardiovascular organ tissue.

29. The method of claim 27, wherein the cutting member comprises a tapered cutting blade.
